(19) United States
(12) Patent Application Publication
(10) Pub. No.: US 2002/0004632 A1
(43) Pub. Date: Jan. 10, 2002

(54) System and method for determining neuronal morphology and effect of substances thereon

(76) Inventors: W. Brent Lindquist, East Setauket, NY (US); Ying Ying Koh, Port Jefferson Station, NY (US); Karel Svoboda, Huntington, NY (US)

Correspondence Address: Jeffrey S. Steen, DILWORTH & BARRESE, LLP, 333 Earle Ovington Boulevard, Uniondale, NY, 11553, US

(21) Appl. No.: 09/829,629

(22) Filed: Apr. 10, 2001

Related U.S. Application Data
(60) Provisional application No. 60/196,080, filed on Apr. 10, 2000.

(51) Int. Cl. A61B 6/00

(52) U.S. Cl. 600/431

(57) ABSTRACT

A system and method provides automated detailed analysis of microscopic neuronal cell morphology. Accordingly, the effects of various substances on the structure and function of neurons can be evaluated based on morphology of the neurons. An algorithm is presented which utilizes a geometric approach for automatically detecting and quantifying the three-dimensional structure of dendritic spines from stacks of image data acquired using microscopy. Results are presented on the measurement of dendritic spine length, volume, density, and shape classification for both static and time-lapse images of dendrites. The approaches presented here are generalizable to other aspects of neuronal morphology.

### PRIORITY

This application claims priority to a U.S. Provisional Application filed on Apr. 10, 2000 having U.S. Provisional Application Serial No. 60/196,080; the contents of which are incorporated herein by reference.

### GOVERNMENT RIGHTS

This invention was funded, at least in part, under grants from the Department of Energy, Nos. DEFG0292ER14261 and DEFG0290ER25084. The Government may therefore have certain rights in the invention.

### BACKGROUND OF THE INVENTION

1. Technical Field

The present invention relates to the study of neurons including neuronal development and the effects of various agents on neurons through analysis of optical imagery.

2. Description of Related Art

Recent large-scale genome sequencing and expression analysis have uncovered a multitude of genes implicated in brain development, learning and memory, regeneration, and neurological diseases. Determining the function of these genes and other substances necessitates advanced techniques for controlling the timing and location of gene expression, combined with specific assays of neuronal cell function or morphology.

Neurons are known to include dendrites, cell bodies and axons. The areas between adjacent neurons are known as synapses. In most synapses presynaptic axons terminate on dendritic spines. Spines are small bulbous compartments consisting of a spine head attached via a thin neck to the dendritic shaft. Spine necks dfffusionally isolate spine heads from their parent dendrites, e.g., spines restrict the diffusion of Ca²⁺ and other second messengers. In addition, spines contain a variety of organelles. Spines contain post-synaptic densities, one of the most complex signaling assemblies, which include synaptic receptors and their regulators as well as various structural and adhesion molecules. Spines also contain machinery required for protein translation.

Spine growth is associated with synaptogenesis. During periods of synaptogenesis dendrites grow filopodia, relatively long actin rich protrusions that often make several synapses; later these filopodia are typically replaced by mature spines. Molecular mechanisms underlying spine genesis and stabilization are beginning to be investigated. Since actin is highly enriched in dendritic spines, most studies have focused on pathways associated with the regulation of actin dynamics. Calcineurin appears to be important in actin stabilization in spines. The Rho family of small GTPases, including Rho, Rac, and Cdc42, regulates various aspects of the actin cytoskeleton and also modulates dendritic structure and spine density.

Recent experiments have revealed that important aspects of cognitive function, such as experience-dependent plasticity, neural integration and learning and memory are correlated with variations in dendritic branching morphology, and with spine density and distribution. Similarly, age-related deficits in short-term memory, important forms of neural dysfunction have been localized, in part, to dendrites and spines.

Recently direct measurements in mammalian brain slices have revealed that synaptic plasticity can manifest itself in sprouting of filopodia, and spines, in an N-methyl-D-aspartate receptor (NMDA-R) dependent manner. On the other hand prolonged NMDA-R activation leads to a loss of spines. Other studies have shown that spine density and shape is controlled by background electrical and synaptic activity. For example, brief exposure to the sodium channel blocker TTX increases the density of dendritic spines. Spines display subtle actin-based motility that appears to be abolished by low levels of alpha-amino-3-hydroxy-5-methyl-4-isoxalone (AMPA) or glutamate. Low levels of AMPA also block lesion-induced spine degeneration. Thus, spines are stabilized by low levels of activation of synaptic receptors, but grow in response to a global reduction of activity, as well as strong focal increase in activation of synaptic receptors. The signaling mechanisms underlying this complex response are not understood.

These and other findings have motivated extensive efforts to obtain quantitative descriptions of dendritic and spine morphologies, both statically and dynamically. Due to its superior resolution capability in revealing ultrastructures at synaptic junctions, serial section electron microscopy (SSEM) has been used to quantify dendritic spine structures in three-dimensions (3-D). This is, however, a non-vital form of observation and an extremely labor-intensive histological approach requiring the physical sectioning of the tissue into very thin sections and detailed manual and/or semi-automatic registration and outlining of the structures on each serial section.

Modem fluorescence microscope methods, such as confocal laser scanning microscopy (CLSM) and two-photon excitation laser scanning microscopy (2PLSM) offer many advantages over SSEM, at the expense of reduced resolution. Sectioning is achieved by limiting the detection (CLSM) or excitation (2PLSM) of fluorescence to a sub-femtoliter focal volume. Optical imaging is rapid and noninvasive. The exquisite selectivity of fluorescence allows the detection of even single molecules against a background of billions of others. Optical microscopy thus occupies a unique niche in biology due to its ability to perform observations in intact, living tissue at relatively high resolution. The properties of fluorescence microscopy images are well understood. To image neuronal structure, neurons are labeled with fluorescent molecules that fill the cytoplasm homogeneously. Voxel values report the convolution of the density of fluorescent probes with the point-spread function (PSF) of the imaging system, which is essentially equal to the focal volume and is easily measured. Studies of morphological plasticity based on CLSM and 2PLSM measurements of spine length and density have been described.

Despite these advances in modem imaging techniques, the analysis of neuronal structure has remained largely manual. The considerable amount of time and effort required for manually extracting spine measurements has precluded routine studies of large amounts of data. In addition, results are not precisely reproducible as accuracy is dependent on the skill and habituation of the user. A few detection and estimation techniques (Rusakov et al., Quantification of dendritic spine populations using image analysis and a tilting dissector, J. Neurosci. Methods, 1995; 60: 11-21; Watzel, et al., Detection of Dendritic spines in 3-dimensional images, DAGM-Symposium Bielefeld, 1995; 160-167; Herzog, et al., Restoration of three-dimensional quasi-binary images from confocal microscopy and its application to dendritic trees, Cogswell C J, Conchello J. and Wilson, T., editors, Three-Dimensional Microscopy: Image Acquisition and Processing IV. SPIE Proceedings, 1997; Kilbom et al., Delineating and tracking hippocampal dendritic spine plasticity using neural network analysis of two-photon microscopy, Soc. Neurosci. Abstr., 1988; 24: 422-425) of varying degrees of automation have been suggested to overcome the tedium and improve accuracy and reproducibility of the result, none of which has apparently been used and verified on large data sets. Rusakov, et al., supra, applied a medial axis construction (skeletonization) to 2-D dendritic images to obtain spine length measurements in 2-D and estimated the corresponding 3-D measurements using a stereological sampling procedure. As the medial axis is sensitive to surface features, manual screening of the medial axis was required to select among spine, dendrite and irregular surface-induced features (i.e. artifacts). Since measurements were based solely on the medial axis, no volumetric estimates were obtainable. Watzel, et al., supra, have also suggested the detection of dendritic spines using medial axis based identification. Their 3-D algorithm was restricted to images containing a single dendrite. The dendrite backbone (‘centerline’) was extracted from the medial axis and the remaining medial axis ‘spurs’ branching off the backbone were used to identify candidate spines. A length tolerance was employed to distinguish true spines from artifact ‘spurs’. No further analysis beyond that for a single dendritic image was presented. Herzog, et al., supra, employed a 3-D reconstruction technique using a parametric model of cylinders with hemispherical ends to ‘fit’ the shape of the dendrites and the spines. In this method, short spines or spines with thin necks were hard to detect and had to be manually added to the model. An approach using neural network recognition for spines (Kilbom, et al., supra) has also been suggested.

The need to understand the structure and function of neurons is a continuing one. Similarly, there is a continuing need to determine the effects of various substances on the development, structure and function of neurons.

### SUMMARY OF THE INVENTION

An algorithm for determining neuronal structure by analyzing a microscopy image is provided wherein the algorithm includes a processing module for processing the image and extracting neuronal structures therefrom based on geometrical features of the neuronal structures, and an analyzing module for analyzing the extracted neuronal structures to determine at least one characteristic thereof.

Also provided is a method for determining the effect of a substance on a neuron which includes subjecting the neuron to the substance, imaging the neuron to generate at least one image, subjecting the at least one image to an algorithm which contains (i) a processing module for processing the image and extracting neuronal structures therefrom based on geometrical features of the neuronal structures and (ii) an analyzing module for analyzing the extracted neuronal structures to determine at least one characteristic thereof, and comparing the at least one characteristic to a corresponding at least one characteristic of a control neuron. In addition, dual-color images can be created wherein one color is used to determine the structure of dendrites and spines and another color is used to measure the distribution of various cellular proteins.

### BRIEF DESCRIPTION OF THE DRAWINGS

FIGS. 1 (a)-(c) illustrate a raw microscopy image, and deblurred microscopy images after 5 and 20 iterations;

FIGS. 2(a)-(b) illustrate a medial axis from a segmented image of a dendritic image with arrows indicating loop (L) formed by overlapping spines, separate skeletons for disconnected features (D) and spurious cell debris, and two backbones extracted from this medial axis;

FIGS. 3(a)-(c) are diagrams illustrating a projected view of 12 spine candidates (shaded gray) with d_{b}(S)=12, a sketch indicating distances and spine candidates used in the spine detection algorithm of the present invention; and an ideal spine candidate symmetric along the line SE;

FIG. 4 is an illustration in 2-D of the orientation criterion used to determine if detached (D) or attached (A) spine components need to be merged;

FIGS. 5(a)-(b) provide a comparison of manual and automatic spine detection on a segment of a hippocampal CA1 dendrite, respectively;

FIG. 6 provides a comparison of manual and automatic measurements of individual spine length (left), average spine length (center) and spine density (right) for the dendrite shown by FIGS. 5(a)-(b);

FIG. 7 illustrates charts of spine volume-length scatterplots according to determined spine type for all spines in experiments E₁ and E₂;

FIGS. 8(a)-(b) illustrate charts showing the number of spines detected in the image at each time step and the detection history of 52 spines followed for 25 minutes, e.g., 1 spine was detected in each image taken over the 25-minute period, where spine 14 was seen sporadically over the entire period;

FIGS. 9(a)-(b) illustrate charts showing measured distributions of spine length and volume as a function of time for the population of 52 spines followed in a time-series of images, and measured distribution of spine motility index fitted to an exponential decay function; and

FIG. 10 illustrates charts showing lengths of five spines plotted as a function of time showing comparison between manual (close circles) and automated (open circles) measurements.

FIG. 11(A) depicts three deblurred 2PLSM images of neurons labeled with GFP. Left image shows neurons cotransfected with wild-type mTOR kinase; center image shows GFP transfected control neurons; right image shows neurons cotransfected with an inactive mTOR kinase mutant.

FIG. 11(B) graphically illustrates the results of analysis using an algorithm according to the present invention which allows comparison of spine density of GFP transfected neurons, mTOR transfected neurons and inactive mTOR transfected neurons.

FIG. 12(A) depicts three deblurred 2PLSM images of neurons labeled with GFP. Left image shows neurons cotransfected with neuroligin (NLG); center image shows GFP transfected control neurons; right image shows neurons cotransfected with an NLG mutant designated AChE.

FIG. 12(B) graphically illustrates the results of analysis using an algorithm according to the present invention which allows comparison of spine density and spine length of GFP transfected neurons, NLG transfected neurons and AChE transfected neurons.

### DESCRIPTION OF PREFERRED EMBODIMENTS

The present invention provides efficient, detailed automated analysis of axonic, dendritic and spine morphologies, thus allowing assessment of the effects of various genes and other substances on neurons. Spines participate in cell-to-cell contact and their growth and retraction is powered by actin based motility. The signaling networks underlying spine responses are controlled by neurotransmitter receptors and neural activity. Naturally and artificially induced perturbations in the signaling networks result in alterations in axonic, dendritic and spine morphologies and dynamics which can be measured according to the present invention with speed and a degree of accuracy and consistency previously unknown.

An automatic dendritic spine detection and analysis algorithm appropriate for 3-D images obtained via laser scanning microscopy or other type of microscopy is presented. The algorithm of the present invention uses a geometric approach; it is highly automatic and contains only a few parameter settings. It can be applied to static images as well as time-series images. There is no limitation on the number or the structure of the axons or dendrites in the image. In addition to spine length and density, volumetric measurements and spine classifications are obtained using the algorithm. Finally, a simple extension of the algorithm allows the measurement of distribution of proteins in dendrites and spines.

The automatic dendritic spine detection and analysis algorithm of the present invention offers an objective and consistent analysis, requiring minimal amount of supervision and makes accessible 3-D morphological characterizations of spine length, volume, shape classification and spine density. Comparison of results on spine length and density between the manual and automatic approach of the present invention on a large number of samples have validated the automatic approach for both static and time-lapse, i.e., time-series, images.

The automated analysis greatly enhances speed, consistency and objectivity. The timing results provided below show that automated analysis of time-lapse data consisting of 50 images tracking a total of 30-50 spines takes about 4 hours CPU time on a Pentium™ III 500 Hz processor, whereas for manual analysis, an experienced user will typically take 12 to 16 hours.

For static images the time savings compared to manual analysis may not be as significant if experimental conditions vary so significantly that new parameters have to be determined for each image to be analyzed. However, automated analysis using the algorithm of the present invention still provides more detailed, complete and objective quantification than manual analysis.

The automatic algorithm of the present invention assumes that the spines are simply connected to the dendrites. Small looping structures in the medial axis indicate pairs of spines that are too close to be resolved by imaging or segmentation. If it satisfies the protrusion criterion each such structures will be detected as a single entity. Resolution of such structures as paired spines is currently not implemented. These occurrences are estimated to affect less than 2% of the spine population.

The inventive algorithm enables calculation of spine volume, previously not possible with manual analysis. Volumetric measurements offer insight into understanding the electrical capacity of the spines and the structural and electrophysiological properties of neuronal dendrites. It is therefore an important parameter for characterizing dendritic spines. Spine volume has not been reported previously in any of the prior art automatic methods. The volume measurements agree reasonably well with an SSEM analysis of similarly aged animals, even though the preparation techniques for the specimens are entirely different.

The various morphology-based measurement capabilities presented below allows application of the investigation of the functional significance of dendritic spines and their plasticity to a wide spectrum of experimental and pathological conditions. Automatic morphometry significantly improves the scale and accuracy of such studies. Although not absolutely essential to the present invention, in certain embodiments, it may be assumed that the image to be analyzed is of a bi-phase medium, with one phase being the neuronal cytoplasm (dendritic phase), the other being the background tissue.

In accordance with the present invention, a substance is contacted with and/or inserted into a neuron and the effects of the substance on morphology is evaluated based on the observations relating to spine length and density, volumetric measurements and spine classifications according to the present algorithm. After a neuron is analyzed by the present algorithm, it may be compared to a control neuron to determine the differences, if any, that are associated with administration of a substance of interest to the neuron. A control neuron includes any neuron used for comparison purposes. For example, a neuron which has been subjected to a substance of interest and an imaging dye can be compared to (i) a neuron containing that imaging dye or another imaging dye only, (ii) a neuron containing that imaging dye or another imaging dye, and another substance of interest, or (iii) a neuron containing that imaging dye or another imaging dye, and a different concentration of the substance of interest.

Substances which can be investigated include any known material which is amenable to be applied to a neuron. Such substances include, but are not limited to, chemical and/or physical agents such as microorganisms, DNA, RNA, proteins, peptides, carbohydrates, lipids, drugs, radiation, temperature, pH, and diagnostic agents. For example, genes, growth factors, enzymes, hormones, metals, viruses, bacteria, toxins, dyes, electromagnetism, gamma radiation, neurotransmitters, electrolytes, vitamins, minerals, antibiotics, anesthetics, antivirals, antiseptics, antimicrobials, antiinflammatories, analgesics, steroids, calcium channel blockers, antiarrhythmics, psychotropics, antidepressants and the like may all be contacted with or inserted into neurons for evaluation. The substance may be known, or not known, to have physiological effects since one aim of the present invention is to determine whether or not a substance has any effect on a neuron.

Methods for subjecting neurons to a substance (including causing a substance to enter into neuronal cells) are well known. For example, passive or active diffusion utilizing concentration gradients (osmolarity), solubilizers, permeation enhancers such as aprotic solvents, e.g., DMSO, may be utilized to effect entry.

In a preferred embodiment, genetic engineering techniques are utilized to impart substances into neurons. “Transformation” or “transfection” (used interchangeably herein) of neuronal cells refers to delivery of nucleic acid (DNA or RNA)) into a neuron by any method. Suitable expression vectors include, but are not limited to plasmids, cosmids, phage, phagemids, artificial chromosomes and the like. Transfection of host cells can be accomplished by, e.g., electroporation, viral transfer, lipid mediated transfer, calcium phosphate precipitation, direct injection and biollistic transfer to name a few.

For example, electroporation is suitable for introducing macromolecules, including, but not limited to, DNA, RNA, dyes, proteins and other various chemical agents, into neuronal cells. Electroporation refers to the permeabilization of cell membranes by application of short duration electric field pulses, traditionally between relatively large plate electrodes. During the electric pulse, charged macromolecules, including DNA, are actively transported by electrophoresis across the cell membrane through these pores. Noncharged molecules can also enter the pores by passive diffusion. Upon pulse termination, pores reseal over hundreds of milliseconds as measured by recovery of normal membrane conductance values.

Biollistic transfer is a preferred method for transfection herein and refers to any method for introducing foreign molecules into a cell using velocity driven microprojectiles such as tungsten or gold particles. Such velocity driven methods typically originate from pressure bursts which include, but are not limited to, helium driven, air driven, and gunpowder driven techniques. In biollistic gene transfer, a desired substance, e.g., a plasmid containing a nucleic acid sequence of interest, is precipitated on polymeric or metallic beads. Indeed, combinations of genes can be delivered by precipitating multiple plasmids onto beads. Thus, for example, neurons can be transfected with DNA encoding red fluorescent protein to determine cell structure, a chimera between a gene of interest and DNA encoding green fluorescent protein to track distribution of the gene product and a third plasmid of a specified function. Biollistic gene transfer also allows accurate control over the number of plasmids which may be introduced into the target cell which allows a measure of control over the amount of target gene products in the cell.

Thus, oliogonucleotides, chimeric genes, fusion proteins, ligands, receptors, molecular labeling systems such as fluorescent molecules, radiolabels, antibodies, antigens, avidin, streptavidin, biocytin, and biotin are examples of substances which are suitable for transfection herein. Examples of fluorescent molecules (fluorochromes), include green fluorescence protein (GFP), color shifted mutants of GFP including red shifted mutants, yellow shifted mutants and blue shifted mutants, amino coumarin acetic acid (AMCA), fluroscein isothiocyanate (FITC), tetramethylchodamine isothiocyanate (TRITC), Texas Red, Cy3.0, Cy5.0 and dextran conjugates of fluorochromes. Such labels may be used independently or coupled to other molecules such as antibodies, antigens, avidin, streptavidin, and nucleic probes. It is contemplated that DNA or RNA encoding fluorescent proteins or other labeling moieties may be fused to DNA or RNA encoding a desired protein prior to transfection of such a chimeric molecules into the neuronal cell.

The ability to transfer multiple genes into cells is helpful in studies of the interaction between different proteins. Transferring genes for colored proteins, such as green fluorescent protein, has proven immensely useful for labeling cells in order to visualize their shapes. These dyes can also be attached to or otherwise coadministered with other proteins in order to see where these proteins are located and move within the cell. As demonstrated below, transfection was used to fill brain cells with protein dyes in order to observe cell morphology. By introducing other genes along with the gene for a fluorescent protein, the effects of various proteins on neuronal cell growth can be observed.

In a preferred embodiment, the neurons being evaluated are studied in preparations that are as intact as possible. Accordingly, while individual isolated neurons can be evaluated using the methods described herein, a living brain slice offers an attractive compromise between the limitations of cultured neurons (e.g., limited synaptic plasticity) and the experimental difficulties of working with intact animals. For example, brain slices may be cultured on membranes according to procedures described in Maletic-Savatic et al., Science, 283, pp. 1923-1927 (1999) and Stoppinin et al., J. Neurosci. Methods, 37, pp. 173-182 (1991), the contents of each being incorporated herein by reference. Neurons in cultured brain slices preserve many of the aspects of neural functions including normal membrane properties, spine morphologies and robust synaptic plasticity. It should be understood, however, that the methods of the present invention are also applicable to use in primary cell cultures.

Any suitable technique involving microscopy to adequately image neurons known to those skilled in the art may be utilized in accordance with the present invention. Serial section election microscopy, confocal laser scanning microscopy (CLSM) and two-photon excitation laser scanning microscopy (2PLSM) may be used. A preferred embodiment involves 2PLSM since it allows imaging neurons in intact neural tissues. 2PLSM is well-suited to image fluorescent molecules. Sectioning may be achieved by limiting the exitation of fluorescence to a subfemtoliter volume. 2PLSM allows imaging of small fluorescent molecules, e.g., GFP, filled structures such as dendritic spines and axon terminals, even in intact brain nervous tissue. 2PLSM also allows detection of submicron spine length and density changes involved in synaptic plasticity in brain slices and the intact brain. Furthermore, 2PLSM allows measurement of movement of molecules with submicron resolution. 2PLSM also allows the excitation of two fluorophores with different emission wavelengths with the same excitation wavelength, facilitating the simultaneous measurement of neuronal structure (in one fluorescence channel) and the distribution of selected proteins (detected through a second fluorescence channel).

The ability to assess the effects of substances on neurons according to the present invention provides an efficient modality to determine what gene products or combinations of gene products are involved in the structure of axons and/or dendrites. Indeed, structure/function relationships may be uncovered and evaluated according to the present invention. For example, mutants of naturally occurring proteins can be evaluated for their effects on neurons, e.g., dominant negative and constitutively active forms of enzymes. Genes that control particular functions in neurons can be identified and studied by transfecting with libraries of transgenes and breaking the libraries into smaller pools. Moreover, the effects of pharmacologic agents on neurons can evaluated according to the present invention.

The description below describes the algorithm used to analyze 3-D scanning microscopy images of fluorescent neuronal structures (Section A). The images collected are analyzed to include, but are not limited to, detection and measurement of dendrites; dendritic spines; synapses; distributions of subcellular components using chimeric proteins; and synaptic function using genetically encoded fimctional probes. The algorithm includes a processing module and an analyzing module for performing the following steps.

An image is first processed by deconvolution and the dendritic phase extracted (Section A.1). The dendrites are identified via their backbones (Section A.2), which are extracted from a medial axis construction. Spines are tiny appendages attached to dendrites, and because of their small size, they can often only contain a few dye molecules and thus show only dim fluorescence. They are adjacent to larger, brighter dendrites and they therefore are to detected against a hazy background. Unlike the prior art, the spines are not detected from the medial axis branches emerging from the backbone as it is difficult to distinguish true spines from artifacts by this procedure. Instead, according to the present invention, the spines are detected as geometric protrusions relative to the backbone (Section A.3).

Each protrusion is subjected to a protrusion criterion to distinguish true protrusion from non-spine dendrite surface irregularity. As very thin necks are too weak to be detectable, some spine “heads” appear to be separated from the dendrite and are detected as detached components. After initial detection, a search is implemented to associate detached components with their appropriate bases. For time-series data, in which the same dendritic branch is imaged over a sequence of time intervals, translational effects in time are corrected for and individual spines are then traced (Section A.4) through the time ordered sequence of images. Finally, morphological characterizations of the population of detected spines are extracted (Section A.5).

The steps described in Sections A.1-A.4 are preferably performed by the processing module, while the steps described in Section A.5 are preferably performed by the analyzing module of the automatic dendritic spine detection and analysis algorithm

An imaging setup and the biological preparation used to obtain data for testing and verification of these algorithms are summarized in Section B and the Examples herein. Results of the application of these algorithms to the analysis of hippocampal CA1 neurons and a small number of hippocampal CA3 neurons are presented in the Examples as well.

A. Image Analysis

A.1 Image Deconvolution and Segmentation

The intrinsic spatial resolution limits of optical microscopy arise from the diffraction of light; light from a point source is ideally imaged to a larger spot characterized by the Airy finction. The measured spread resulting from a given optical setup is referred to as the point-spread-function (PSF). As a result, the intensity recorded in any voxel (volume element) of a digitized image is a convolution of intensities from its neighborhood.

Deconvolution is used to correct aspects of the image degradation due to the PSF. A variety of deconvolution techniques are available which employ either theoretical or experimental measures of the PSF. In addition, blind deconvolution methods can be employed which, concurrently with the deconvolution, reconstruct an estimated PSF of the image. However, the presence of noise and the band-limited nature of the PSF limit the improvements by means of classical deconvolution techniques. Therefore some blurring will remain even after deconvolution due to a trade-off between sharpening of the image and noise amplification.

In addition, the photomultiplier tube (PMT) detectors used in most laser scanning microscopes are “noisy”. Even in darkness PMTs produce spontaneous bright pixels which may be referred to as “shot noise”. One can deal with shot noise by applying a median filter to the image. Median filters are known in the art. See, e.g., Tukey, Exploratory Data Analysis, Addison-Wesley, Reading, MA (1971). For example, the median filter may be a non-linear, lowpass filter which replaces the greyscale value of each voxel v in the digitized image by the median greyscale value of v and its 26 neighbors. This effectively removes shot noise but not real spines which, under typical magnifications employed, have an effective width covering many voxels.

A prior art iterative reblurring deconvolution algorithm was applied to the median filtered image, which requires either a theoretical or experimentally measured PSF. See, Kawata and Ichioka, J. Opt. Soc. Am., 1980; 70:762-772, the contents of which are incorporated herein by reference. Briefly, iterative reblurring proceeds as follows. Let o⁽⁰⁾ (x, y, z) denote the experimental 3-D image and h (x, y, z) an appropriate PSF. Let * and ¤ denote the convolution and correlation operators. The deconvolved image ô^{(k)} (x, y, z) in the k-th iteration is

ô^{(k)}=^{(k-1)}+{o⁽⁰⁾¤h−ô^{(k-1)}*(h¤h)}.

A non-negativity constraint is applied to ô^{(k)} at the end of each iteration.

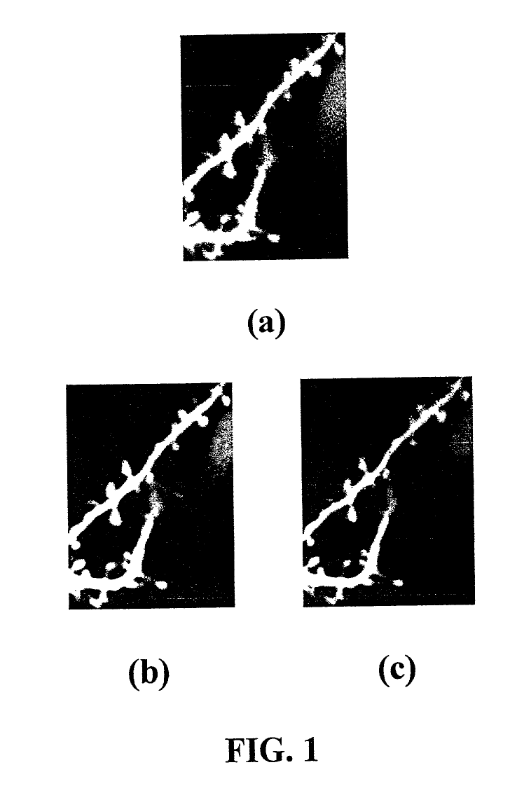

For the images analyzed in the Examples herein, the PSF was measured by imaging a number of sub-resolution microspheres and averaging their individual PSFs to reduce noise. FIG. 1 demonstrates the result of iterative reblurring a raw image (FIG. 1(a)) after 5 (FIG. 1(b)) and 20 (FIG. 1(c)) iterations. Preferably, one employs kₘₐₓ=5 iterations of deblurring. It should be understood that those skilled in the art may use other deconvolution algorithms for reblurring.

Segmentation is a generic imaging term for labeling each voxel in a greyscale (or color) image with an integer identifier designating its “population type”. For dendritic morphometry, this requires distinguishing neuron voxels from the background tissue voxels. A large number of segmentation algorithms are available. See, e.g., Pal and Pal, Pattern Recognition, 1993; 26; 1277-1294. As the dendritic images are processed first by median filtering and deconvolution, simple thresholding is used for this final segmentation step; all voxels of intensity greater than a threshold value are identified as neuron, otherwise as background. In general, a trade-off between selection of dim spines and reduction of noise on the dendrite surface is made in selecting the threshold.

A.2 Dendritic Backbone Extraction

Geometric analysis of a 3-D irregularly shaped object is difficult; such analyses typically employ models based upon geometrically simple “unit” objects. The algorithm of the present invention builds upon the medial axis algorithm disclosed by Lee et al., “Building skeleton models via 3-D medial surface/axis thinning algorithms,” CVGIP: Graph. Models Image Process., (1994); vol. 56: 462-478, to provide a skeleton from which the backbone of each dendrite in an image can be extracted.

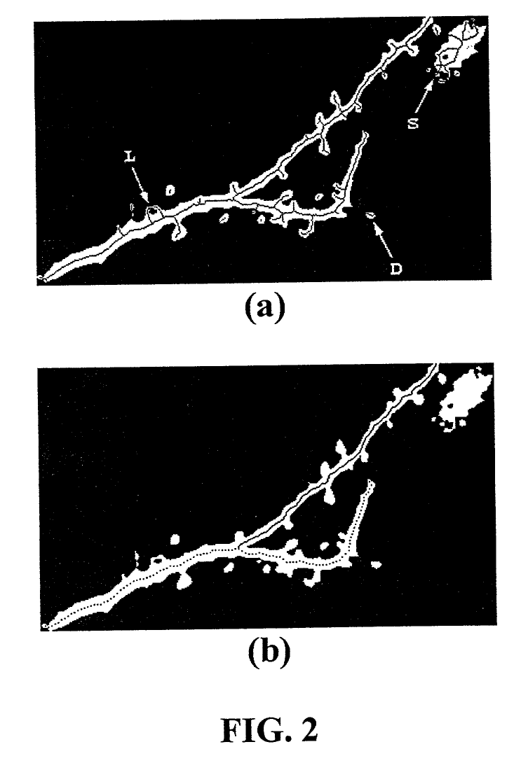

Intuitively, the medial axis captures a geometrically faithful skeleton (consisting of curve segments joining at vertices) of an object. In a digitized image these curve segments consist of linked sequences of voxels, with the vertices being voxels at which these segments join together. An example of the medial axis of a portion of a segmented dendritic image is shown in FIG. 2(a). (This is a view perpendicular to the optical axis.) The medial axis obtained for the dendritic phase contains the backbone (“centerline”) of each dendrite as a subset.

In addition to the backbone however, the medial axis contains “spurs” and other features which correspond to spine-related or non-spine-related surface features (e.g., incipient dendritic branches); to surface artifacts resulting from digitization effects, segmentation errors, and boundary effects due to the finite imaged volume; or to spurious cell “debris”. Due to resolution limits, spines emerging near each other may appear to have overlapping tips in the digitized image, resulting in the appearance of small loops in the medial axis (top of the parent branch in FIG. 2(a)). A separate skeleton for each disconnected component of dendritic phase is also contained in the medial axis.

From the medial axis, the backbone for each dendrite is extracted (FIG. 2(b)) in two steps. The first step is achieved by removing the medial axis segments corresponding to all disconnected dendritic components, and trimming short spurs and loops on the medial axis. Removal of long “spurs” is problematic as they may correspond to filopodia or incipient branches of the dendrite. These long “spurs” are dealt with in the next step.

In the second step, a backbone is traced through each dendritic branch employing a decision based upon minimum deviation angle whenever a vertex on the trimmed skeleton is encountered. If necessary, the number, n, of dendrites in the image can be specified so that only the n longest backbones are retained. Any remaining medial axis segment that is not part of a traced backbone removed. The final set of dendritic backbones extracted from FIG. 2(a) is shown in FIG. 2(b).

A.3 Spine Detection

With the dendritic backbones isolated, the spine detection algorithm of the present invention proceeds in four steps: detection of detached dendritic phase components (Section A.3.a); detection of attached spine components (Section A.3.b); elimination of spurious or incomplete spine components (Section A.3.c); and merging of spine components (Section A.3.d). Since a spine may be composed of one or more detached pieces and possibly an attached base in the segmented image, the identification of any spine is not finalized until all four steps have been completed.

A.3.a Detached spine component detection

Dendritic phase components disconnected from the backbone-containing dendrites are detected and tentatively identified as detached spine components. For each detached spine component a record is kept of its center of mass, the closest dendritic backbone voxel and the dendritic surface voxel lying on the line joining the center of mass to the backbone voxel. Detached dendritic phase components that are further from the nearest dendrite surface voxel than a maximum distance tolerance are interpreted as false positive signals and ignored. For the images analyzed in the Examples herein, the length tolerance is 6 μm. It should be understood that the length tolerance can be adjusted when appropriate.

A.3.b Attached spine component detection

Ignoring the detached spine components, every dendrite phase voxel v is labeled with a distance, d_{b}(v), to its closest backbone voxel. Thus tips of protrusions on the dendrite surface are assigned the largest distances. These tip voxel locations are then processed in descending order of d_{b}.

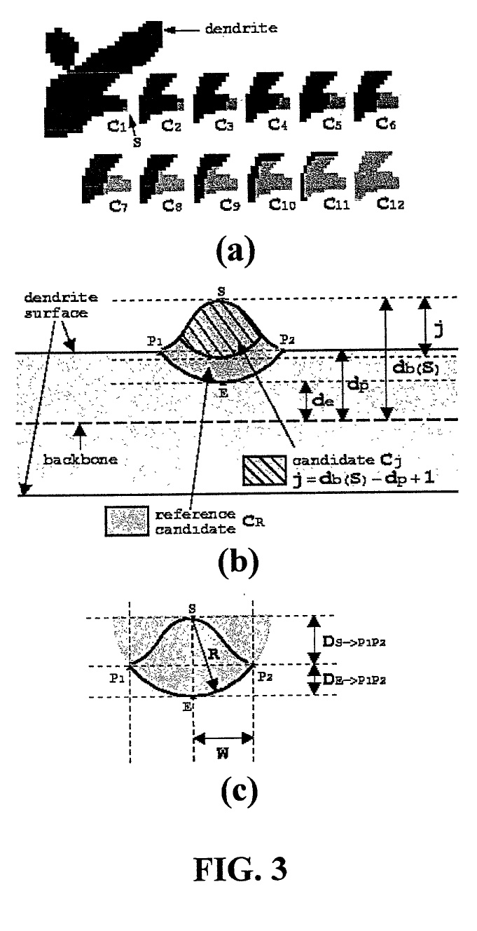

For each tip voxel S, a sequence {Cᵢ}, i=1, . . . ,d_{b}(S), of candidate spines is generated. Candidate Cᵢ consists of all voxels w whose distance d_{S} (w) from S is d_{S}(w). FIG. 3(a) shows a 2-D projected view of the candidates (gray voxels) C₁, . . . ,C₁₂ for a tip S having d_{b}=12. The smaller candidates clearly contain insufficient voxels to correctly represent the spine, whereas the larger candidates protrude too far below the dendrite surface. The optimal choice of a spine candidate would terminate at the surface of the dendrite. This is achieved by estimating for the local thickness of the dendrite as explained below.

To estimate the local thickness and choose the optimal candidate, a ring of “spine-surface boundary points” are determined for each candidate. For clarity of explanation, the algorithm is first illustrated in 2-D assuming the image is projected onto its focal plane. The 3-D algorithm is described afterwards. Two “surface boundary voxels” P₁ and P₂ on the 2-D projection are shown in FIG. 3(b). For each candidate Cᵢ, along with the two surface boundary voxels P₁^{C}ᵢ and P₂^{C}ᵢ, a “base voxel” E^{C}ᵢ having the furthest penetration into the dendrite is also determined. An idealized sketch of such a projection illustrating S, P₁, P₂ and E is shown in FIG. 3(b). These surface points are used to determine the best measure of the dendrite thickness as follows. A “reference candidate” C_{R} is selected to be the smallest volume candidate with the minimum surface to backbone distance, dₚ≡minᵢ{d_{b}(P₁^{C}ᵢ), d_{b}(P₂^{C}ᵢ)}. A reference candidate C_{R} and the distance d_{P} are illustrated in FIG. 3(b).

In an ideal case 2 dₚ is the width of the dendrite and the best candidate for the spine would be Cᵢ where j=d_{b}(S)−dₚ+1. In practice, spines and dendrite surfaces in the images are quite irregular and additional criteria must be satisfied before a final candidate is accepted. To be accepted as a true spine, the candidate is required to satisfy a heuristic protrusion criterion. Let D_{S→P}_{₁}^{_{P}}_{₂}^{C}ᵢ denote the perpendicular distance from S to the line segment

$$\overline{{{P}_{1}}^{{C}_{i}} {{P}_{2}}^{{C}_{i}}}$$

of Cᵢ and let D_{E→P}_{₁}^{_{P}}_{₂}^{C}ᵢ denote the perpendicular distance from the base voxel E^{C}ᵢ to the line segment

$$\overline{{{P}_{1}}^{{C}_{j}} {{P}_{2}}^{{C}_{j}}}$$

as illustrated in FIG. 3(c). The spine candidate Cⱼ is required to satisfy

D_{S→P}_{₁}^{_{P}}_{₂}^{C}ᵢ≧D_{E→P}_{₁}^{_{P}}_{₂}^{C}ᵢ (1)

Criterion (1) clearly requires that favorable spine candidates protrude out further from the dendrite surface than they protrude into it. It however favors spines with narrow necks, as the following argument shows. Consider an ideal, symmetric spine whose base is the arc of a circle with radius R as sketched in FIG. 3(c). (Clearly i plays the integer role of the radius for the digitized candidates Cᵢ.)

Let 2 W denote the distance between P₁ and P₂. Simple trigonometry gives

$$\begin{matrix}{D}_{E \to {P}_{1} {P}_{2}} = R - \sqrt{{R}^{2} - {W}^{2}} , & ( 2 ) \\ {D}_{S \to {P}_{1} {P}_{2}} = \sqrt{{R}^{2} - {W}^{2}} . & ( 3 )\end{matrix}$$

Then the protrusion criterion (1) requires R≧2W/\sqrt{3} in order to accept a spine candidate, favoring narrow-necked spines and rejecting wide-necked spines. Since R will increase faster than W for true spines (i.e., i increases faster than the distance

$$\overline{{{P}_{1}}^{{C}_{i}} {{P}_{2}}^{{C}_{j}}} ) ,$$

instead of testing only the candidate Cⱼ for the protrusion criterion, a range of candidates Cᵢ are tested for which i is close to j. Specifically, candidates having values i in the range d_{b}(S)−dₚ≦i≦d_{b}(S)−(dₚ+dₑ1)/2, where dₑ≡d_{b}(E^{C}_{_{R}}), are considered. If one of the candidates Cᵢ with i value in this range satisfies (1), then the candidate Cⱼ is accepted as a true spine; otherwise the protrusion is rejected as a spine.

The 3-D algorithm proceeds in a similar fashion except that instead of using the projected pair of surface boundary points, the entire ring of surface boundary points in 3-D are considered. The minimum “surface points to backbone” distance is used to find the reference candidate C_{R} to correct for the local thickness of the dendrite. The distance from S (or E) to the ring of voxels is calculated by measuring the perpendicular distance of S (or E) to the plane that best fits the ring of voxels in 3-D.

A.3.c Component Elimination

Spines touching the boundary of the imaged region are ignored as they are incomplete. This is also a useful technique for eliminating “debris” and other axons or dendrites in the background of the image that are near or touching the dendrites of interest. The inventive algorithms are written to allow for the imposition one or more non-overlapping polygonal areas on the plane of the image slices. The interior of the union of these polygons is regarded as the region of interest for the spine detection algorithm; any structure exterior to the polygons is ignored. By setting the polygonal edges to cross through unwanted structures they are also automatically ignored. As mentioned, detached components further from the dendrite surface than a maximum distance are also eliminated.

A.3.d Component Merging

As a spine may be identified from multiple detached “head” and attached “base” components, a final merging algorithm which accounts for the position and orientation of all possible spine pieces is performed. The merging algorithm considers every component, checking for possible merges with other components. Any merged entity is reconsidered as a new single component, and rechecked for possible further merges.

Merging can occur between two detached (DD) components (the merged entity is still considered a detached component) or between detached and attached (DA) components (the merged entity is then considered to be an attached component). Two criteria are employed for DD or DA type merging.

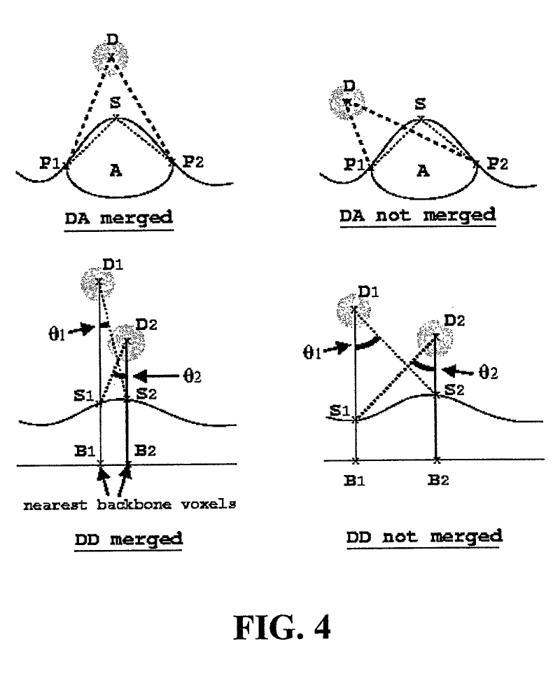

The first criterion is maximum separation; the two components to be merged are required to be close enough (a center-of-mass to center-of-mass separation≦3 μm). The second criterion requires appropriate relative orientation of the two components as demonstrated in 2-D in FIG. 4. For DA type merging, the tip S of the attached component A is required to lie within the triangle DP₁P₂. (In 3-D, the tip S is required to lie within the cone determined by D and the ring of spine-surface boundary points.) For DD type merging, the average angle subtended by the center of mass of each spine with the surface voxel locations of both spines is required to be less than 30°.

A.4 Image Registration and Spine Tracing

A time sequence of 3-D images must be registered to correct for possible translational movement of the specimen. After registration, individual spines are then traced and identified through the image sequence.

Each consecutive pair of images Fᵢ and Fᵢ₊₁ are co-registered using the spines separately identified in each image. The offset \vec{o}=(oₓ, o_{y}, o_{z}) of Fᵢ₊₁ with respect to Fᵢ is allowed to vary within a window |oₓ|≦wₓ, |oₓ|≦wₓ, |oₓ|≦wₓ,|o_{y}|≦w_{y},|o_{z}|≦w_{z}. Only integer voxel offsets are considered. A conventional registration method maximizes the cross correlation of two images; thus no decision can be made until the correlation arrays are computed for all offsets. Instead, an efficient sequential search method, as disclosed by Bamea and Silverman, 1972 in “A class of algorithms for fast image registration,” IEEE Trans. Computers, 1972, vol. C-21, 179-186, is utilized which computes the l₁ norm (absolute value sum) image difference

ε(\vec{o})=ΣᵢΣⱼΣₖ|Fᵢ(i,j,k)−Fᵢ₊₁(i−oₓ,j−o_{y},k−o_{z})|.

over all offsets \vec{o} in the window for which ε(\vec{o}) is less than a predetermined threshold value T. The offset \vec{o} with minimum ε(\vec{o}) provides the optimal registration. In practice, wₓ=w_{y}=w_{z}=5 voxels, and T is the average number of total spine voxels in Fᵢ and Fᵢ₊₁.

Individual spines are traced through the time-series. Two spines at different times are considered to be the same if their percentage overlap (measured in voxels) is larger than 25% of the volume of at least one of them.

A.5 Morphological Characterization

Spine length, density and volume are computed in accordance with one aspect of the present invention. Spines are also classified according to their shape.

For a detached spine (without any attached component), the spine length is determined by the distance from the recorded dendrite surface voxel (corresponding to the associated dendrite) to the furthest spine voxel (corresponding to the detached spine) from the dendrite. For spines that are fully or partially attached (consisting of a base and one or more detached components) to the dendrite, the spine length is determined by the distance from the center of mass of the base boundary points to the furthest spine voxel (possibly detached from the dendrite). For the images analyzed in the Examples herein, the automated spine length measurement is calculated from a 2-D projection. The reason for this is that the manual spine analysis measurements against which the automatic analysis results are to be compared are performed in 2-D by projecting the 3-D stack of image slices along the optical direction.

Spine density is computed as the number of spines per unit length of dendritic backbone. For purposes of comparison with manually analyzed images which are analyzed in 2-D projection only, backbone length is also measured from a 2-D projection onto the slice plane.

Spine volume is measured according to the intensity values of the deconvolved greyscale image. For 2PLSM, the excitation of fluorescence is limited to a sub-femtoliter focal volume (≈0.5×0.5×1.5 μm³) which is larger than that of individual spines. The intensity value recorded for each voxel in a spine is a sum of the fluorescence from all dye molecules excited within the focal volume. The maximum intensity voxel near the center of a spine is therefore a measure of the volume of a spine. As the larger cross-sectional areas of a dendrite are typically larger than the maximum cross-sectional area of the focal region, the maximum voxel intensity recorded along the dendrite backbone is a measure of the size of the focal volume, assuming the fluorescence is saturated near the center of the dendrite. See, Svoboda et al., Science, 272, pp. 589-593 (1996) and Sabatini and Svoboda, Nature, 408, pp. 589-593 (2000). The spine volume is defined as the ratio of the maximum spine intensity to the maximum dendrite intensity multiplied by an empirically determined focal volume,

$$Spine Volume = \frac{Maximum Spine Intensity}{Maximum Dendrite Intensity} \times Focal Volume .$$

The following classification of spine shapes is used stubby, thin, mushroom. Spine shape is decided based on spine length (L), head diameter (dₕ) and neck diameter (dₙ). In general terms for thin spines, spine length should be much greater than the neck diameter (L>>dₙ). For mushroom spines, spine length should not exceed neck diameter by more than a factor of 5, and the head diameter should be much greater than the neck diameter (dₕ>>dₙ). For stubby spines, the neck diameter is approximately equal to the length of the spine. The specific criteria adopted in this classification utilize the ratios L/dₙ and dₕ/dₙ to classify theirshape as summarized in Table 1.

TABLE 1
Ratio criteria for the classification of stubby,
thin and mushroom spines.

| | dₕ/dₙ | | |
|---|---|---|---|
| L/dₙ | [0,1.3) | [1.3,3) | [3,∞) |
| [0,2/3) | stubby | mushroom | mushroom |
| [2/3,2) | stubby | stubby | stubby |
| [2,3) | stubby | mushroom | mushroom |
| [3,5) | thin | mushroom | mushroom |
| [5,∞) | thin | thin | thin |

A.6 Measurement of the distribution of fluorescent proteins.

Segmentation is performed on data from one fluorescence channel. The other fluorescence channel can be used to measure the distribution of protein components, such as chimeric proteins linked to GFP. The analysis is simply to measure the fluorescence on the second channel as a measure of protein concentration, in pixels that were previously determined to belong to the neuronal structure.

B. Image Acquisition

From a data analysis standpoint CLSM and 2PLSM provide essentially equivalent challenges. However, to gain an understanding of the dynamics of neuronal circuits, neurons should preferably be studied in preparations that are as intact as possible. For many questions of sub-cellular physiology, as stated above, the living brain slice offers an attractive compromise between the obvious limitations of cultured dissociated neurons and the experimental difficulties encountered when working with intact animals.

One problem with brain slice physiology has been that scattering of light makes traditional optical microscopies, including CLSM, difficult in living tissues. For these reasons, as also stated above, the data is preferably collected using 2PLSM, which allows high resolution fluorescence imaging in brain slices up to several hundred microns deep with minimal photodamage.

At present, parameters that require routine adjustments include the region of interest and segmentation threshold. Other parameters that are used in the deconvolution, backbone extraction, spine component elimination and tracing algorithms are empirically determined; they remained the same throughout all of the examples that have been described herein. Segmentation is crucial to the analysis due to the relatively low intensity associated with small spines and the high intensity of the dendrites. Choosing a critical threshold is important; simple thresholding is adequate for most images that have been preprocessed by median filtering and deconvolution.

It is contemplated to provide the algorithm of the present invention within a server accessible via a network, such as the Internet or a local area network (LAN), by a plurality of users. The users can then transmit data to the server for processing using the algorithm. The results would then be transmitted by the server to the user via e-mail or by other known techniques.

The following examples are included for purposes of illustrating certain aspects of the present invention and are not intended to limit the invention as defined by the claims herein.

### EXAMPLE 1

### Sample Preparation and Microscopy

Cultured hippocampal brain slices were prepared from 7 day old rats. After five days in vitro a small subset of neurons were biollistically transfected (for example, Lo, et al., (1994). Neuronal transfection in brain slices using particle-mediated gene transfer, Neuron 13, 1263-1268, the contents of which are hereby incorporated by reference) with a plasmid carrying the gene for enhanced green fluorescent protein (GFP) (commercially available from Clonetech) At least two days after transfection, slices were transferred to a perfusion chamber for imaging. Labeled neurons were identified and imaged using a custom-made 2PLSM laser scanning microscope (as described in Mainen, Z. F., Maletic-Savatic, M., Shi, S. H., Hayashi, Y., Malinow, R., and Svoboda, K. (1999), Two-photon imaging in living brain slices, Methods 18, 231-239, the contents of which are hereby incorporated by reference). The light source was a Ti:sapphire laser running at a wavelength of ≈990 nm (repetition frequency 80 MHz; pulse length 150 fs). The average power delivered to the backfocal plane of the objective (40x, NA 0.8) varied depending on the imaging depth (range 30 to 150 mW). Fluorescence was detected in whole-field detection mode with a photomultiplier tube.

### EXAMPLE 2

### Static Analysis

To validate the automatic spine detection algorithm, an experiment, E₁, involving ≈200 spine measurements over 15 dendrites of hippocampal CA1 and a small number of CA3 neurons was performed. The same imaged regions were subjected to both automatic and manual analysis. A total of 174 spines were identified by both methods; an additional 10 spines were identified only by the manual method; and a further 28 spines were identified only by the automatic method.

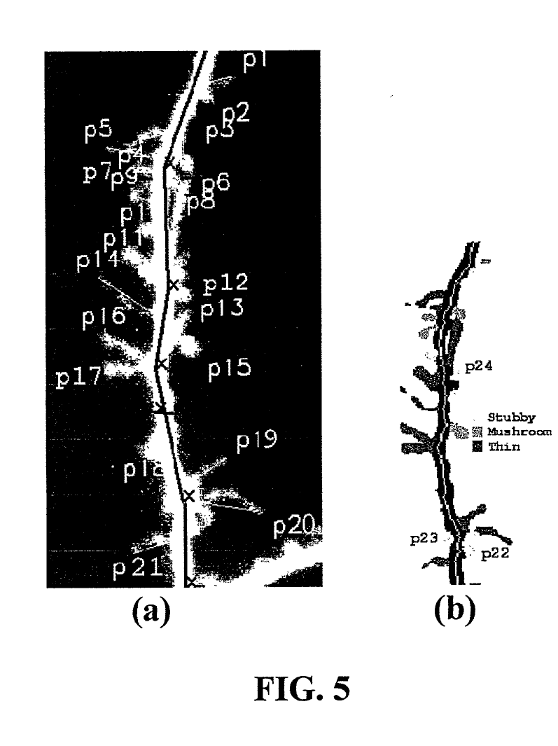

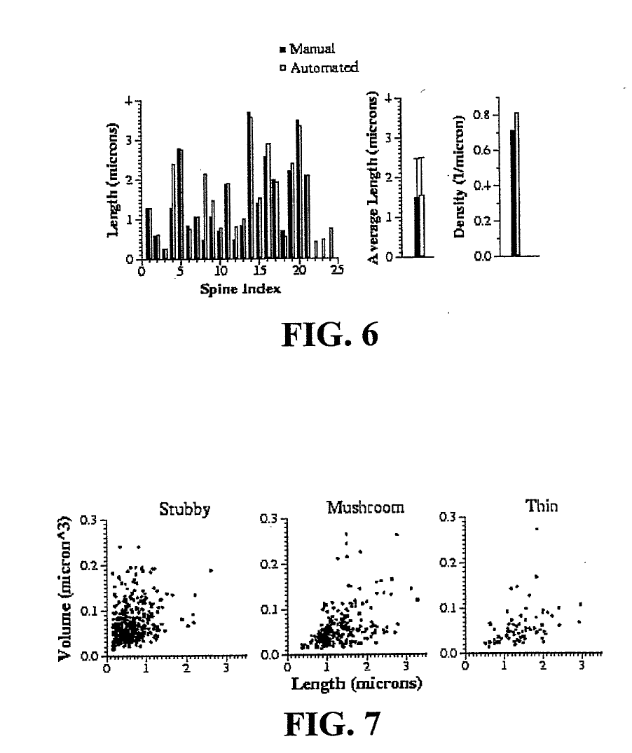

The results of the manual and automated analysis for one of the dendrites in this experiment are illustrated in FIG. 5(a)-(b). Twenty-one spines were detected by both methods; three additional, relatively short, spines were detected by the automatic method. FIG. 6 compares the individual spine lengths, average spine length and spine density measured for this particular dendrite. Spines 8 and 22 demonstrate the difficulties encountered by both detection methods when two spines appear to overlap. For spine 8, the manual detection has identified only the shorter of two spines which appear to overlap; whereas the automatic method has identified only the longer. For spine 22, again two spines appear to overlap and are considered as a single spine by the automatic method. On the other hand, the manual method failed to identify either of them.

TABLE 2
Measured mean spine lengths
(± standard deviation) for the spines in experiment E₁.

| | | Mean spine length |
|---|---|---|
| Population size | Method | (μm) |
| 174 | Manual | 1.05 ± 0.62 |
| 174 | Automatic | 1.08 ± 0.63 |
| 10 | Manual | 0.75 ± 0.57 |
| 28 | Automatic | 0.38 ± 0.28 |

Table 2 compares the mean spine length measured by each method for the population of 174 spines detected in common. The mean lengths for those spine detected by only one of the methods are also presented. For the common detected spine population, the manual and automatic spine length measurements agree to within one standard deviation, though the standard deviations are large. (The large standard deviation is partly due to averaging over spines of different shape classification.) A paired samples Student's t-test to determine whether the difference in measurements by the two methods is significant provides a stronger test of the agreement between the two methods of length measurement. Column 1 of Table 3 summarizes the results of the paired t-test; there is no significant difference between the two methods of length measurement.

TABLE 3
Paired samples t-test results for
measured spine lengths and densities, experiment E₁.

| | Spine length | Spine density |
|---|---|---|
| Degrees of freedom | 173 | 14 |
| t statistic | −1.24 | −0.87 |
| p-value | 0.22, two-sided | 0.40, two-sided |

A one-way ANOVA was used to test for any dependence of dendrite origin on the observed differences in measured spine length. The test produces an F statistic value of 0.95 (dₙᵤₘ=14 and d_{den}=159) with a p-value of 0.51. Thus the differences in spine length measurements between the two methods are uniform across the different dendrites.

The mean spine length for the 28 spines detected only by the automatic method of the present invention indicates a population of smaller length spines. The results in Table 4 of an independent samples t-test for these 28 spines shows that the difference in spine lengths is significant compared to that obtained by either measurement method for the population of 174 commonly detected spines. The results indicate that the automatic algorithm of the present invention is detecting short spines more consistently than the manual method.

TABLE 4
Independent samples t-test results, experiment E₁.

| Method (population | Manual (n = 174) | Automatic (n = 174) |
|---|---|---|
| Automatic only | df = 200 | df = 2 |
| (n = 28) | t = 5.63 | t = 5.82 |
| | p = 6 × 10⁻⁸, two-sided | p = 2 × 10⁻⁸, two-sided |
| Manual only | df = 182 | df = 182 |
| (n = 10) | t = 1.51 | t = 1.65 |
| | p = 0.13, two-sided | p = 0.10, two-sided |

For the 10 spines detected only by the manual method, the mean length measurement lies midway between that obtained for the common and automatic only populations. An independent samples t-test (Table 4) shows no significant differences with the measurements obtained by either method for the 174 commonly detected spines. The results (df=36, t=2.65, p=0.01, 2-sided) of an independent samples t-test between these 10 spines and the 28 detected only by the automatic algorithm indicate a significant difference between the spine lengths of these two populations.

Visual observation of these 10 spines reveals that 7 of them touched the boundary of the image region and were consequently rejected by the automatic algorithm. The remaining 3 were not resolved by the automatic algorithm as each touched some neighboring spine (which was detected). This is thus a reflection of the combined effectiveness of the median filter, deconvolution and simple thresholding algorithms in segmenting the images. Based upon visual investigation of the images, no more than 2% of the spines were estimated not to have been resolved by the automatic algorithm due to segmentation related effects.

TABLE 5
Measured mean spine density
(± standard deviation for the dendrites in experiment E₁.

| | | Mean spine density |
|---|---|---|
| Population size | Method | (μm⁻¹) |
| 15 | Manual | 0.45 ± 0.09 |
| 15 | Automatic | 0.47 ± 0.15 |

Table 5 compares the mean spine densities separately measured by each method for the 15 dendrites. (For the manual method this is a total population of 184 spines; for the automatic method, 202 spines.) The density measurements by either method agree to within one standard deviation. For the paired sample of 15 dendrites, a Kolmogorov-Smimov test shows that the dendrite by dendrite difference in the automatic and manual measured densities is very close to normal, so that a paired-dendrite samples t-test can be applied. Column 2 of Table 3 summarizes the result; the automatic spine density measurement is not significantly different from the manual.

### EXAMPLE 3

### Static Analysis

For spine volume measurement and shape classification, automated results are reported herein as no manual determination is available. A second experiment, E₂, was performed under the same experimental conditions to increase the sample size (E₁+E₂) up to 700 spines. The spine volumes were calculated from the ratio of the maximum intensity values of the spine to the dendrite, as described in Section A.5 using an empirically determined focal volume of 0.5×0.5×1.5 μm³. FIG. 7 shows the volume-length correlation plot for the spines measured in experiments E₁ and E₂ according to their determined classification. The mushroom shaped spines occupy the widest spectrum of lengths and volumes. The ratio of stubby:mushroom:thin spines is 0.54:0.36:0.10.

TABLE 6
Comparison of automated spine length and volume measurements of
hippocampus cells in PND 7 cultured neurons with the PND 15
SSEM results of Harris et al., “Three-dimensional structure of dendritic
spines and synapses in rat hippocampus (CA1) as postnatal day 15 and
adult ages: implications for the maturation of synaptic physiology and
long-term potentiation,” J. Neurosci. 1992, vol. 12, 2685-2705.

| | | Shape classification | | |
|---|---|---|---|---|
| Measurement | Method | stubby | mushroom | thin |
| volume (μm³) | automatic | 0.07 ± 0.04 | 0.06 ± 0.05 | 0.06 ± 0.04 |
| | SSEM | 0.11 ± 0.07 | 0.18 ± 0.09 | 0.05 ± 0.03 |
| length (μm) | automatic | 0.65 ± 0.37 | 1.35 ± 0.55 | 1.38 ± 0.54 |
| | SSEM | 0.65 ± 0.38 | 0.95 ± 0.30 | 1.40 ± 0.39 |

Table 6 summarizes the average spine volume and length measurements in each shape category and presents comparison with the SSEM results (Harris et al. 1992; Table 4) on rat hippocampus CA1 cells for postnatal day (PND) 15 animals. All automatic measurements are within 1.5 standard deviations of the SSEM result, though the volume results are generally smaller. It is noted however that the automatic results come from cultured neurons and younger aged animals. In addition, no corrections for any fixation-induced changes have been performed in the SSEM study.

### EXAMPLE 4

### Time series analysis

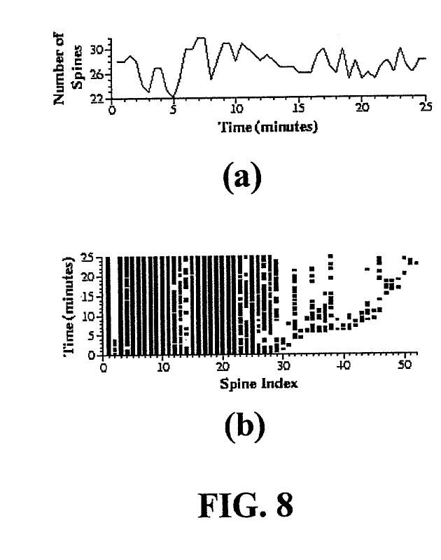

Time-series data provides the ability to capture dynamic changes in dendritic spine morphology. A series of 50 3-D images was taken at 30 second intervals spanning a time period of 25 minutes. FIG. 8(a) shows the number of spines detected in the images as a function of time using the automatic method. On average 27.5±2.3 spines were detected in each image. An interest lies in the question of the frequency of observation of any particular spine over time.

In total, 52 spines were detected and traced through the time-series. FIG. 8(b) shows how the observations of the 52 spines were distributed in time. The spines were indexed (1→52) according to the first time in which they appeared. Thus, 27 spines were observed over the full 25 minutes of image taking; among those, 16 were present at all time points.

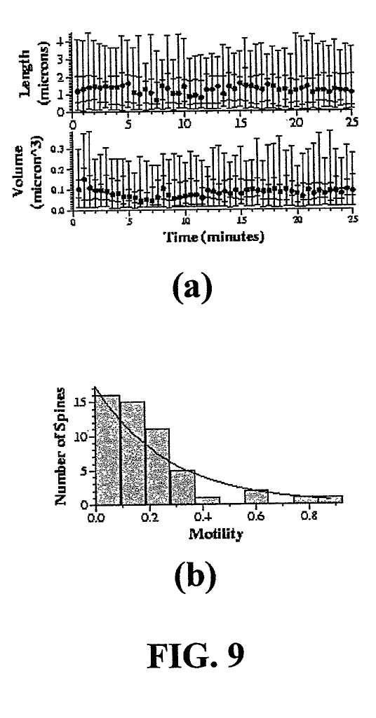

FIG. 9(a) summarizes the distribution of spine length and volume as a function of time. Both length and volume distributions are skewed, with smaller lengths and volumes dominating, consistent with the stubby, mushroom, and thin spine ratios noted above. The dynamics of the spines are measured using an index for spine motility, which is defined as the summed difference in length of a spine in time divided by the total number of time steps. FIG. 9(b) plots the distribution of motilities for the 52 spines traced in this series. For this limited data set, the number of spines (n) decreases with motility (m) approximately as n(m)=n(0)e^{-3.69m}.

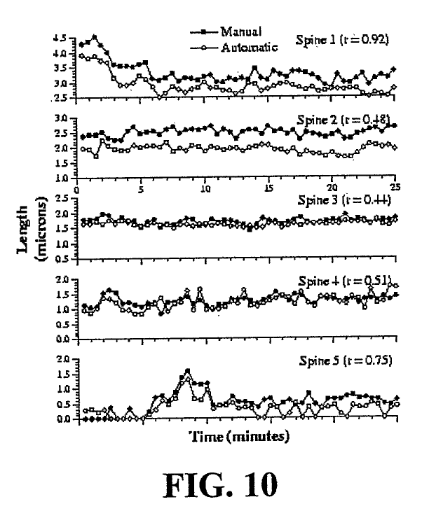

A comparison between automated and manual length measurements was made for a limited subset of this time series data; manual length measurements were made on a subset of 10 of the 52 spines. FIG. 10 presents comparisons between the automated and manual spine length measurements as a function of time for 5 of the spines chosen to represent different average lengths. Consistently longer lengths were measured by the manual method for the longer spines (1 and 2). For the medium length spines (3 and 4), the manual and automated results are very similar. For the short spine (5), some deviations are observed; occasionally the spines were not detected by the automatic method.

A paired t-test was performed on this subset of 10 spines to determine whether the average spine length determined by the automated measurement is significantly different from that determined by the manual measurement. A Kolmogorov-Smimov test shows that this sample of 10 difference measurements (−0.32±0.17 μm) is very close to normal so that a paired samples t-test can be applied. The observed t statistic value is −2.79, with p=0.02, two-sided, revealing some significance in the averaged difference.

While this is contrary to the results in experiment E₁, it is noted that the manual measurements were made by a different user than in E₁. Pearson correlation was therefore used to test whether the automatic and manual measurements are correlated in time. The correlation values (r) obtained for the 10 spines for n=50 time points range from 0.92 to 0.29; with two-sided p-values ranging from 0.00 to 0.04. Thus, for these 10 spines significant correlations between the automatic measurements and the manual measurements in time are observed. Therefore, the existence of a systematic bias between the manual and automated measurements for this set of data that can be attributed to a change in the user making the manual measurements. The significant Pearson correlation however indicates that the manual measurements are duplicating the trends found by the automatic measurements.

### EXAMPLE 5

### Evaluation of the Effects of mTOR Kinase on Spine Formation

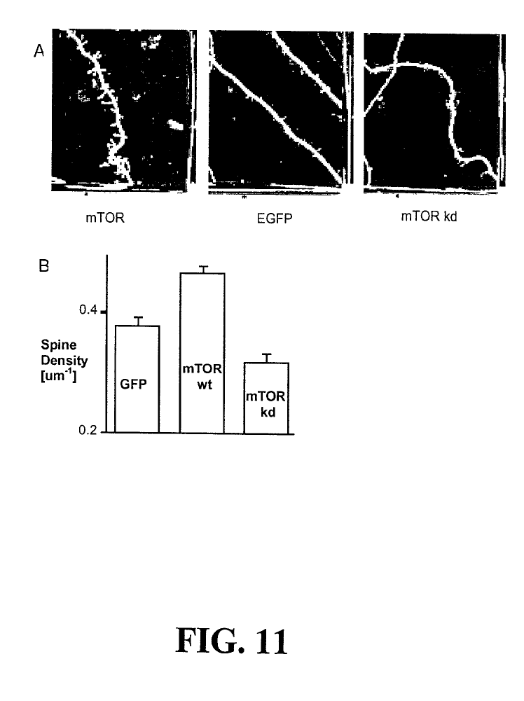

mTOR kinase controls the phosphorylation of the translation regulators p70^{56k} and 4E-BP1. It is a central regulator of cell growth and highly expressed in dendrites and highly associated with synaptic proteins. Rat hippocampal CA1 neurons were transfected by biollistic gene transfer with GFP alone or together with the wild-type kinase (mTOR wt) or a non-functional mutant (mTOR kd) that acts as a dominant negative (for details of plasmids see: Sabatini et al., (1999). Interaction of RAFT1 with gephyrin required for rapamycin-sensitive signaling, Science 284, 1161-4, the contents of which are hereby incorporated by reference) (for methods see Example 1). The 3d structure was imaged using 2PLSM as described in Example 1 and the inventive algorithm utilized to analyze dendritric shape. As can be seen in FIGS. 11(A) and 11(B) mTOR kinase controls spine size and density, i.e., mTOR wt increased spine density and mTOR kd decreased spine density as compared to the GFP control. Data from at least 5 neurons were in every group.

### EXAMPLE 6

### Evaluation of the Effects of Overexpression of Neuroligin on Spine Formation

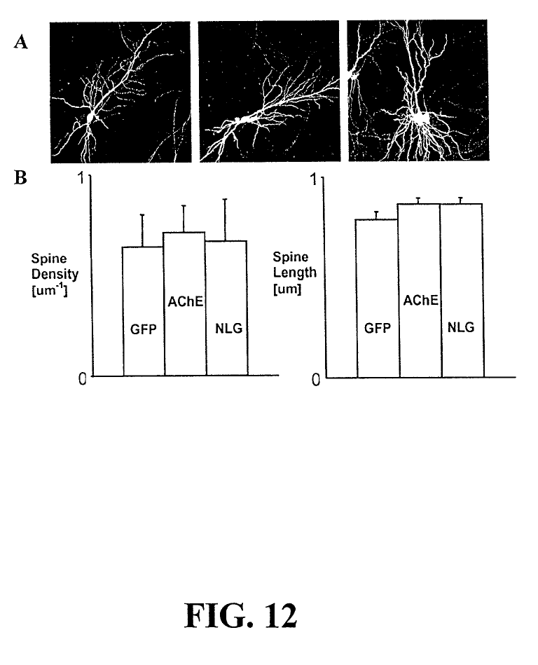

Neuroligin (NLG) is believed to be involved in synapse formation. When nonneuronal cells are engineered and cultured to express NLG, they are associated with the development of presynaptic structures in contacting axons, suggesting that NLG-neurexin interactions are a key step in synapse formation. Rat hippocampal CA1 neurons were transfected by biollistic gene transfer with GFP alone or together with NLG or a mutant form of NLG that does not contain the AchE domain (designated “AChE”) (See, Scheiffele et al., (2000). Neuroligin expressed in nonneuronal cells triggers presynaptic development in contacting axons, Cell 101, 657-69, the contents of which are hereby incorporated by reference). The 3d structure was imaged using 2PLSM as described in Example 1 and the inventive algorithm utilized to analyze dendritic shape. As can be seen in FIGS. 12(A) and 12(B), overexpression of NLG did not produce a detectable phenotype change in spine density or spine shape. Data from at least 5 neurons are in every group.

### EXAMPLE 7

### Measurement of the distribution of GluR1-GFP in dendritic spines.

Glur1 is a synaptic receptor that is thought to play an important role in synaptic plasticity. To measure the distribution of Glur1 in dendritic spines neurons were transfected with a virus expressing Glur1-GFP (for methods see Shi, S. H., Hayashi, Y., Petralia, R. S., Zaman, S. H., Wenthold, R. J., Svoboda, K., and Malinow, R. (1999). Rapid Spine Delivery and Redistribution of AMPA Receptors After Synaptic NMDA Receptor Activation, Science 284, 1811-1816, the contents of which are hereby incorporated by reference). Neurons expressing glur1-GFP were then patch-clamped and filled with a red fluorohore (Texas Red, Molecular Probes). A two color image was then acquired using 2PLSM, with an excitation wavelength of 910 nm. The red image was used for segmentation and to perform a spine analysis. The green image was used to estimate the distribution of Glur1-GFP in dendrites and spines.

It will be understood that various modifications may be made to the embodiments and examples disclosed herein. For example, alternative algorithms can be created to accomplish the criteria set forth above. It should be understood that, notwithstanding the emphasis on spine morphology, the algorithms described herein can be applied to the larger axonic, dendritic and cell body structures of neurons to determine length, volume, shape classification and density. Therefore, the above description should not be viewed as limiting, but merely as exemplifications of preferred embodiments. Those skilled in the art will envision other modifications within the scope and spirit of the claims appended hereto.

1. An algorithm for determining neuronal structure by analyzing a microscopy image, said algorithm comprising:
a processing module for processing the image and extracting neuronal structures therefrom based on geometrical features of the neuronal structures; and
an analyzing module for analyzing the extracted neuronal structures to determine at least one characteristic thereof.

2. The algorithm according to claim 1, wherein the image is selected from the group consisting of static image and time-series images.

3. The algorithm according to claim 1, wherein the processing module performs a deconvolution process to extract the neuronal structures.

4. The algorithm according to claim 3, wherein the extracted neuronal structures include a plurality of dendrites which are identified via their respective backbones.

5. The algorithm according to claim 4, wherein the processing module detects from the plurality of dendrites a plurality of spines as geometric protrusions relative to the backbones.

6. The algorithm according to claim 5, wherein the processing module subjects each geometric protrusion to a protrusion criterion to distinguish geometric protrusions associated with the plurality of spines from geometric protrusions not associated with the plurality of spines.

7. The algorithm according to claim 6, wherein the processing module correlates each detached spine of the plurality of spines to its respective dendrite of the plurality of dendrites.

8. The algorithm according to claim 5, wherein the analyzing module analyzes each of the plurality of spines to determine the at least one characteristic thereof.

9. The algorithm according to claim 8, wherein the at least one characteristic thereof is selected from the group consisting of spine length, spine density and spine volume.

10. The algorithm according to claim 9, wherein the spine length for a spine detached from its respective dendrite is determined by the distance from a recorded dendrite surface volume element corresponding to the respective dendrite to a furthest spine volume element corresponding to the detached spine.

11. The algorithm according to claim 9, wherein the spine length for a spine fully or partially attached to its respective dendrite is determined by the distance from the center of mass corresponding to base boundary points associated with the fully or partially attached spine to a furthest spine volume element corresponding to the fully or partially attached spine.

12. The algorithm according to claim 9, wherein the spine density is computed as the number of spines per unit length of dendritic backbone.

13. The algorithm according to claim 9, wherein the spine volume is computed by multiplying the ratio of maximum spine intensity to maximum dendrite intensity by focal volume.

14. The algorithm according to claim 5, wherein the analyzing module classifies each of the plurality of spines according to shape.

15. The algorithm according to claim 14, wherein each of the plurality of spines is classified in one of the following classifications: stubby, thin and mushroom.

16. The algorithm according to claim 14, wherein the analyzing module determines the shape of each of the plurality of spines based on spine length, spine head diameter and spine neck diameter.

17. The algorithm according to claim 16, wherein a spine is classified as a thin spine if the spine length is greater than the neck diameter; a spine is classified as a stubby spine if the neck diameter is approximately equal to the spine length; and a spine is classified as a mushroom spine if the spine length does not exceed neck diameter by more than a factor of 5 and the head diameter is greater than the neck diameter.

18. A method for determining the effect of a substance on a neuron comprising:
subjecting the neuron to the substance;
imaging the neuron to generate at least one image;
subjecting the at least one image to an algorithm which contains (i) a processing module for processing the image and extracting neuronal structures therefrom based on geometrical features of the neuronal structures and (ii) an analyzing module for analyzing the extracted neuronal structures to determine at least one characteristic thereof; and
comparing the at least one characteristic to a corresponding at least one characteristic of a control neuron.

19. A method for determining the effect of a substance on a neuron according to claim 18, wherein subjecting the neuron to the substance involves entry of the substance into the neuron.

20. A method for determining the effect of a substance on a neuron according to claim 19, wherein the entry is accomplished by a transfection technique selected from the groups consisting of diffusion, electroporation, viral transfer, lipid mediated transfer, calcium phosphate precipitation, direct injection and biollistic transfer.

21. A method for determining the effect of a substance on a neuron according to claim 18, wherein the image is generated by laser scanning microscopy.

22. A method for determining the effect of a substance on a neuron according to claim 21, wherein the laser scanning microscopy is selected from the group consisting of 2- photon exitation laser scanning microscopy and confocal laser scanning microscopy.

23. A method for determining the effect of a substance on a neuron according to claim 18, wherein the neuron is contained in a brain slice.

24. A method for determining the effect of a substance on a neuron according to claim 18, wherein the image is selected from the group consisting of static image and time-series images.

25. A method for determining the effect of a substance on a neuron according to claim 18, wherein the processing module performs a deconvolution process to extract the neuronal structures.

26. A method for determining the effect of a substance on a neuron according to claim 25, wherein the extracted neuronal structures include a plurality of dendrites which are identified via their respective backbones.

27. A method for determining the effect of a substance on a neuron according to claim 26, wherein the processing module detects from the plurality of dendrites a plurality of spines as geometric protrusions relative to the backbones.

28. A method for determining the effect of a substance on a neuron according to claim 27, wherein the processing module subjects each geometric protrusion to a protrusion criterion to distinguish geometric protrusions associated with the plurality of spines from geometric protrusions not associated with the plurality of spines.

29. A method for determining the effect of a substance on a neuron according to claim 28, wherein the processing module correlates each detached spine of the plurality of spines to its respective dendrite of the plurality of dendrites.

30. A method for determining the effect of a substance on a neuron according to claim 27, wherein the analyzing module analyzes each of the plurality of spines to determine the at least one characteristic thereof.

31. A method for determining the effect of a substance on a neuron according to claim 30, wherein the at least one characteristic thereof is selected from the group consisting of spine length, spine density and spine volume.

32. A method for determining the effect of a substance on a neuron according to claim 31, wherein the spine length for a spine detached from its respective dendrite is determined by the distance from a recorded dendrite surface volume element corresponding to the respective dendrite to a furthest spine volume element corresponding to the detached spine.

33. A method for determining the effect of a substance on a neuron according to claim 31, wherein the spine length for a spine fully or partially attached to its respective dendrite is determined by the distance from the center of mass corresponding to base boundary points associated with the fully or partially attached spine to a furthest spine volume element corresponding to the fully or partially attached spine.

34. A method for determining the effect of a substance on a neuron according to claim 31, wherein the spine density is computed as the number of spines per unit length of dendritic backbone.

35. A method for determining the effect of a substance on a neuron according to claim 31, wherein the spine volume is computed by multiplying the ratio of maximum spine intensity to maximum dendrite intensity by focal volume.

36. A method for determining the effect of a substance on a neuron according to claim 27, wherein the analyzing module classifies each of the plurality of spines according to shape.

37. A method for determining the effect of a substance on a neuron according to claim 36, wherein each of the plurality of spines is classified in one of the following classifications: stubby, thin and mushroom.

38. A method for determining the effect of a substance on a neuron according to claim 36, wherein the analyzing module determines the shape of each of the plurality of spines based on spine length, spine head diameter and spine neck diameter.

39. A method for determining the effect of a substance on a neuron according to claim 38, wherein a spine is classified as a thin spine if the spine length is greater than the neck diameter; a spine is classified as a stubby spine if the neck diameter is approximately equal to the spine length; and a spine is classified as a mushroom spine if the spine length does not exceed neck diameter by more than a factor of 5 and the head diameter is greater than the neck diameter.

40. A method for determining the effect of a substance on a neuron according to claim 18, wherein the substance is selected from the group consisting of nucleic acid, protein, peptide, carbohydrate, lipid, metal, radiation, temperature, pH, drug, toxin, dye, virus, vitamin and mineral.

41. A method for determining the effect of a substance on a neuron according to claim 18, wherein subjecting the neuron to the substance includes exposure of the neuron to at least two dyes such that one dye illuminates the structure of a dendrite and a second dye illuminates distribution of a target molecule in the neuron.

42. A method for determining the effect of a substance on a neuron according to claim 41, wherein the second dye is a fusion protein comprising a fluorescent protein linked to a target protein of interest.
